(19) United States
(12) Patent Application Publication
(10) Pub. No.: US 2021/0139950 A1
(43) Pub. Date: May 13, 2021

(54) MICROSATELLITE INSTABILITY MEASUREMENT

(71) Applicant: Life Technologies Corporation, Carlsbad, CA (US)

(72) Inventors: Harrison Leong, San Francisco, CA (US); Charles Baudo, Oakland, CA (US); Charles Wendell Higdon, III, Pacifica, CA (US)

(73) Assignee: Life Technologies Corporation, Carlsbad, CA (US)

(21) Appl. No.: 17/092,194

(22) Filed: Nov. 6, 2020

Related U.S. Application Data
(60) Provisional application No. 62/932,987, filed on Nov. 8, 2019.
(60) Provisional application No. 62/932,910, filed on Nov. 8, 2019.
(60) Provisional application No. 62/932,752, filed on Nov. 8, 2019.

(51) Int. Cl. C12Q 1/686; C12Q 1/6883; G01N 21/64

(52) U.S. Cl. CPC C12Q 1/686; G01N 21/6428; C12Q 1/6883

(57) ABSTRACT

Systems and methods for detecting microsatellite instability in a biological sample are described. Signal data is received from a capillary electrophoresis genetic analysis instrument, wherein the signal data is measured from fluorescence of fragments comprising nucleic acid sequences amplified from the biological sample via polymerase chain reaction (PCR). The nucleic acid sequences correspond to a plurality of different microsatellite loci and are obtained using a plurality of PCR primers configured to flank a plurality of microsatellite loci of a biological sample. When the PCR primers and the biological sample are combined and subjected to PCR amplification, fluorescently labeled DNA fragments are generated comprising the plurality of microsatellite loci. Fluorescent data obtained from the plurality of fluorescently labelled microsatellite loci are used to classify microsatellite instability of the biological sample.

### CROSS-REFERENCE TO RELATED APPLICATIONS

This application claims the benefit of priority to U.S. Provisional Application No. 62/932,987, filed Nov. 8, 2019; U.S. Provisional Application No. 62/932,910, filed Nov. 8, 2019; and U.S. Provisional Application No. 62/932,752, filed Nov. 8, 2019. The entire contents of these applications, and all other extrinsic materials discussed herein, are hereby incorporated by reference in their entirety.

### BACKGROUND

This disclosure relates generally to DNA fragment analysis.

A causal factor in cancer is thought to be the breakdown of biomolecular machinery to repair DNA. During cell replication, DNA repair mechanisms are critical to the integrity of the replicate cells. When these mechanisms break down, mistakes can accumulate in the DNA carried by the resulting cells. There are cancer fighting drugs that take advantage of this breakdown to identify and destroy tumors. The drugs are most effective when tumors exhibit a high mutation rate which, in turn, is associated with a high degree of malfunction of the DNA repair biomolecular machinery. One way of detecting the circumstances in which the drugs would be most effective is to examine the degree to which DNA deviates from normal at loci where the DNA consists of many repeated subsequences. These subsequences are referred to as micro-satellites.

Microsatellite markers (loci), also known as short tandem repeats (STRs), are polymorphic DNA loci consisting of a repeated nucleotide sequence. In a typical microsatellite analysis, microsatellite loci are amplified by polymerase chain reaction (PCR) using fluorescently labeled forward primers and unlabeled reverse primers. The PCR amplicons are separated by size using electrophoresis. Applications include linkage mapping; animal breeding; human, animal, and plant typing; pathogen sub-typing; genetic diversity; microsatellite instability; Loss of Heterozygosity (LOH); Inter-simple sequence repeat (ISSR); Multilocus Variant Analysis (MLVA); and companion diagnostics for cancer treatments.

When the number of microsatellites at a given DNA locus differs substantially from normal, that microsatellite locus is considered to be microsatellite unstable (MSU). When numerous microsatellite loci exhibit instability, the DNA sample is considered to have high microsatellite instability, MSI high. When there are only a few exhibiting instability, the DNA sample is considered to be MSI low. When none exhibit instability, the DNA sample is considered to be microsatellite stable, MSS.

At a given microsatellite locus, capillary electrophoresis (CE) can be used to measure the number of microsatellites by using fragment analysis. Automated CE uses fluorescent dyes and separates with higher resolution and higher accuracy than other methods such as agarose or polyacrylamide gel electrophoresis.

To run fragment analysis on a CE system, probes and primers can be designed to flank a region of interest. This can be done by attaching fluorescent dyes to primers or probes used with the polymerase chain reaction (PCR) to amplify a DNA locus of interest before the electrophoresis and submitting the amplicons to CE. There is also a sizing standard, a collection of fragments of known sizes labelled with a color that is different than the colors of the test fragments. The labelled PCR products and the sizing standard are then electrokinetically injected into the capillaries. During electrophoresis, the negatively charged DNA fragments moves from the cathode, through the polymer-filled capillary towards the positively charged anode when high voltage is applied between the electrodes.

DNA fragment analysis using CE can be multiplexed, meaning there are multiple fragments in a reaction well going through the same capillary. The smaller fragments usually run faster, and the bigger ones run slower. Shortly before reaching the positive electrode, the fluorescently labelled DNA fragments, separated by size, move through the path of a laser beam. The laser beam causes the dyes on the fragments to fluoresce at different emission wavelengths. A CCD camera detects the fluorescence, and the fluorescence intensities are digitalized, color-coded and displayed as peaks in the electropherogram. Longer fragments will occur later in the data relative to shorter fragments.

### SUMMARY

When the proportion of DNA with microsatellites that differ from the normal molecules is low, it can be very hard to detect that abnormal DNA molecules are present. More accurate ways are needed to analyze the CE data to resolve the uncertainty enough to reliably distinguish between MSI high and MSI stable at a given DNA locus and to determine whether the overall genetic profile can be considered MSI high or MSI low.

There are possible alternatives to using CE fragment analysis. In a simple example, sequencing technologies can be used to sequence the DNA loci of interest and, through sequence analysis (e.g., counting the number of microsatellites in the sequence), assign MSI status. However, using sequencing technologies or similar approaches other than CE fragment analysis may be disadvantageous. For example, DNA sequence analysis has a limited ability to multiplex data. In addition, the process of DNA sequence analysis takes longer, and the analysis may be more error prone.

Prior art solutions involving manual review of CE fragment analysis data to make MSI status calls tend to be rather time-consuming and an inefficient use of limited manual review time. Embodiments of the present invention discussed herein provide very wide coverage of reasonable methods to automatically make MSI status calls in many cases. The methods described can also be used to assign a confidence metric to the calls, for example, by reporting the proximity of calculated results to decision thresholds, which, in turn, can be used to focus human review efforts on those cases where the automated MSI assessment is less confident.

Embodiments of the invention used to detect microsatellite instability in a biological sample are disclosed. Signal data is received from a capillary electrophoresis genetic analysis instrument, wherein the signal data is measured from fluorescence of fragments comprising nucleic acid sequences amplified from the biological sample via polymerase chain reaction. The nucleic acid sequences correspond to a plurality of different microsatellite loci. Different loci can exhibit different signal characteristics. At a particular locus, a hierarchy of analysis methods can be applied that may also be peculiar to the characteristics of the signal data at that locus. For example, a three-level hierarchy could be described as follows: A first processing algorithm is implemented to obtain a first determination, based on the signal data, regarding instability of one or more first microsatellite loci of the plurality of different microsatellite loci. A second processing algorithm is implemented to obtain a second determination, based on the signal data, regarding instability of one or more second microsatellite loci of the plurality of different microsatellite loci. A third processing algorithm is then implemented to measure microsatellite instability of the biological sample based on at least the first determination and the second determination.

Embodiments of the invention describe a collection of ways to analyze the CE data to determine whether a given DNA locus is abnormal and to determine whether the overall genetic profile, combining results from all loci, can be considered MSI high, MSI low, or MSS. The methods described herein provide a means to automatically make the calls. The methods described can also be used to assign a confidence metric to the calls, for example, by reporting the proximity of calculated results to decision thresholds, which, in turn, can be used to focus human review efforts on those cases where the automated MSI assessment is less confident.

Embodiments of the present invention disclosed herein describe a heterogeneous approach to the analysis ranging from simple thresholds up to utilizing deep learning technologies. The reason for this is that assigning the overall genetic profile to MSI high, MSI low or MSI stable can involve one locus of microsatellites in the DNA up to many loci of microsatellites in the DNA. The complexity of analysis algorithms depends on the nature of DNA replication patterns at the loci chosen. Different loci might be chosen for different cancers since some may be more sensitive to a given cancer type compared to other cancer types and/or, in combination with other loci, may yield a more sensitive and/or specific test for MSI status, and/or the DNA may be more reliably amplified.

### BRIEF DESCRIPTION OF THE DRAWINGS

FIG. 1 illustrates a system in accordance with an embodiment of the present invention;

FIG. 2 illustrates an exemplary capillary electrophoresis process used in some embodiments of the present invention;

FIG. 3 illustrates an exemplary genetic analyzer instrument used in some embodiments of the present invention;

FIG. 4 illustrates an exemplary all-in-one cartridge used in the exemplary genetic analyzer instrument of FIG. 3;

FIG. 5 illustrates four exemplary screenshots of user interface displays used in some embodiments of the present invention;

FIG. 6 illustrates a flow diagram depicting a cloud integration process of the exemplary genetic analyzer instrument of FIG. 3;

FIG. 7 illustrates a flow diagram of a method according to some embodiments of the present invention;

FIG. 8 illustrates a flow diagram of alternate methods according to some embodiments of the present invention;

FIG. 9 illustrates a block diagram of a distributed computer system that can implement one or more aspects of an embodiment of the invention; and

FIG. 10 illustrates a block diagram of an electronic device that can implement one or more aspects of an embodiment of the invention.

While the invention is described with reference to the above drawings, the drawings are intended to be illustrative, and other embodiments are consistent with the spirit, and within the scope, of the invention.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

FIG. 1 illustrates system 1000 in accordance with an exemplary embodiment of the present invention. The DNA fragment analysis processes set forth in embodiments of the present invention start with extracting the DNA from a tissue sample. A plurality of microsatellite DNA loci of interest in one or more nucleic acid samples 111 under investigation may be amplified in a PCR reaction performed in amplification instrument 112 such as a thermal cycler. Exemplary thermal cycle instruments used in some embodiments of the present invention include the Applied Biosystems ProFlex PCR System, the SimpliAmp Thermal Cycler, and other thermal cycler systems manufactured by Thermo Fisher Scientific. The PCR reaction is performed using a wet chemistry kit 110 containing specially designed fluorescently labeled primers to flank the microsatellite loci of interest. Other patent applications describe exemplary embodiments of a wet chemistry kit 110 containing specially designed primers and reagents for use in the PCR reaction performed in amplification instrument 112 in detail, including, but not limited to, co-pending U.S. Patent Applications 62/932,752 (Attorney/TFS Docket No. LT01509PRO) and 62/932,910 (Attorney/TFS Docket No. LT01514PRO), both filed on Nov. 8, 2019; and U.S. Non-Provisional patent application Ser. No. ______ (Attorney/TFS Docket No. LT01514), which claims priority to U.S. Provisional Patent Application 62/932,910 filed on Nov. 8, 2019. Each of these patent applications noted above are herein incorporated by reference in its entirety. System 1000 comprises capillary electrophoresis based genetic analyzer instrument (e.g. a sequencing instrument) 101, one or more computers 103, and user device 108. Exemplary genetic analyzer instruments used in some embodiments of the present invention include the Applied Biosystems SeqStudio Genetic Analyzer by Thermo Fisher Scientific, Models 3500, 3720, and 3130 and similar capillary electrophoresis-based genetic analyzers manufactured by Thermo Fisher Scientific and others.

As shown in an exemplary process set forth in FIG. 2, capillary electrophoresis (CE) is a process 200 used to separate ionic fragments by size. In Thermo Fisher Scientific CE instrumentation used in some embodiments of the invention, an electrokinetic injection is used to inject DNA fragments from solution and into each capillary of a capillary array 201 comprising one or more capillaries. During capillary electrophoresis, the extension products of the PCR reaction (and any other negatively charged molecules such as salt or unincorporated primers and nucleotides) enter the capillary as a result of electrokinetic injection. A high voltage charge applied to the sample forces the negatively charged fragments into the capillaries. The extension products are separated by size based on their total charge. The electrophoretic mobility of the sample can be affected by the run conditions: the buffer type, concentration, and pH; the run temperature; the amount of voltage applied; and the type of polymer used.

Shortly before reaching the positive electrode, the fluorescently labeled DNA fragments, separated by size, move across the path of a laser beam 202. The laser beam causes the dyes attached to the fragments to fluoresce. The dye signals are separated by a diffraction system 203, and a CCD camera detects the fluorescence as shown in 204. Because each dye emits light at a different wavelength when excited by the laser, all colors, and therefore loci, can be detected and distinguished in one capillary injection. The fluorescence signal is converted into digital data, then the data is stored in a file format compatible with an analysis software application.

In general, the data coming out of the CE instrumentation is a series of fluorescent peaks instead of a single peak at the exact size of the amplicon expected for a given number of microsatellites. This is caused by nuances in the amplification of the DNA of interest; “stutter” of the biomolecular machinery involved can result in generating amplicons with a few more or a few less microsatellites in the amplicons than the true number of microsatellites. As a result of this “stutter”, there can be some uncertainty in determining the number of microsatellites and/or in determining whether the number of microsatellites differs from the number expected in normal, non-cancerous tissue.

Adding to the complexity of the signals received from the CE instrumentation, a single dye may be used with several different PCR primers that target different DNA loci. This is done because the instrumentation imposes limitations on the number of different dyes that can be used, and the number of DNA loci of interest may exceed the maximum number of dyes that can be used. If the amplicon sizes are sufficiently different between a group of loci for which the same dye is used on their respective PCR primers, the fluorescent peaks associated with each of the loci would be well separated in the data generated by the CE instrument. As discussed above, in embodiments of the present invention, a CCD camera is used that detects the fluorescence, and the fluorescence intensities are digitalized, color-coded and displayed as peaks in the electropherogram. Longer fragments will occur later in the data relative to shorter fragments. Multiple colors of the fluorescence detected by the CCD camera and color-coding in the electropherogram are utilized in embodiments of the present invention as known to those skilled in the art, although not depicted in the black and white FIGS. 1-10 discussed herein.

Instructions for implementing the CE data analysis algorithms 102 shown in FIG. 1 reside in computer program product 104 which is stored in storage 105 and those instructions are executable by processor 106. One or more patent applications describe exemplary embodiments of CE data analysis algorithms 102 in detail, including, but not limited to, this patent application, and co-pending U.S. Provisional Patent Application 62/932,910 (Attorney/TFS Docket No. LT01514PRO) filed on Nov. 8, 2019, and U.S. Non-Provisional patent application Ser. No. ______ (Attorney/TFS Docket No. LT01514), which claims priority to U.S. Provisional Patent Application 62/932,910 filed on Nov. 8, 2019, each of which as noted above are herein incorporated by reference in its entirety. When processor 106 is executing the instructions of computer program product 104, the instructions, or a portion thereof, are typically loaded into working memory 109 from which the instructions are readily accessed by processor 106. In the illustrated embodiment, computer program product 104 is stored in storage 105 or other non-transitory computer readable medium (which may include being distributed across media on different devices and different locations). In alternative embodiments, the storage medium is transitory.

In one embodiment, processor 106 in fact comprises multiple processors which may comprise additional working memories (additional processors and memories not individually illustrated) including a graphics processing unit (GPU) comprising at least thousands of arithmetic logic units supporting parallel computations on a large scale. GPUs are often utilized in deep learning applications because they can perform the relevant processing tasks more efficiently than can typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application specific), field programmable gate arrays and the like, and combinations thereof. In some embodiments, however, a processor such as processor 106 may be implemented as one or more general purpose processors (preferably having multiple cores) without necessarily departing from the spirit and scope of the present invention.

FIG. 3 shows an exemplary genetic analyzer instrument system 300 that may be used in the system of FIG. 1. In some embodiments of the present invention, exemplary genetic analyzer instrument system 300 comprises an Applied Biosystems™ SegStudio™ Genetic Analyzer instrument system, manufactured by ThermoFisher Scientific Inc., although other genetic analyzer instrument systems similarly capable of performing capillary electrophoresis may be used.

System 300 comprises genetic analyzer instrument 310, all-in-one cartridge 320, and cathode buffer 330. Built into genetic analyzer instrument 310 is touchscreen display 340 and USB port 350. Genetic analyzer system 300 used in some embodiments of the present invention allows multiple fragment analysis and/or sequencing runs on the same plate. Genetic analyzer system 300 is easy to use with integrated cartridge-based system 320 and allows researchers to access and monitor experimental runs as well as view data on the integrated touchscreen display 340, or remotely. The fully connected genetic analyzer, along with the simple cartridge design, can be easily shared by multiple researchers in a lab or facility.

In some embodiments of the present invention, an easy-to-use functional core of the instrument includes a cartridge design that helps maximize efficiency and convenience. For example, the SeqStudio Genetic Analyzer mentioned above utilizes an all-in-one cartridge 320, shown in more detail in FIG. 4, that contains the capillary array 440, polymer reservoir 410, polymer delivery system 420, and anode buffer 430. A laser detection window may also be provided in some embodiments of cartridge 320. Cartridge 320 is removable and, in one embodiment of the invention, can be stored on the instrument for up to four months. In some embodiments of the present invention, each cartridge contains a new polymer unique to the SeqStudio system that allows Sanger sequencing and fragment analysis to be performed with no reconfiguration. In one embodiment of the present invention, the cartridge has four capillaries, and can process samples from either standard 96-well plates or 8-well strip tubes. In some embodiments of the present invention, the cartridge and cathode buffer container include radio-frequency identification (RFID) tags that track the number of injections (cartridge) and length of time on the instrument (cathode buffer container). This allows scientists, using the same instrument, to maintain custody of their own cartridges.

Genetic analyzer instrument system 300 allows real-time monitoring of runs on the SeqStudio Genetic Analyzer. As shown in FIG. 5, Instrument 300 displays results 510 for each capillary in real time. Once an injection is finished, several quality checks are calculated and displayed in exemplary screen display 520. If an injection produces poor traces or poor QC values, those samples can be re-injected, with altered injection parameters, if desired. Exemplary screenshot 530 from an off-site computer monitoring shows the progress of a run. In exemplary screenshot 540 as used in some embodiments of the present invention, runs set up in a PlateManager user interface can be uploaded directly to the instrument. PlateManager allows investigators to assign multiple sequencing and fragment analysis runs on the same plate, taking advantage of the universal polymer in the cartridge and use them only when needed, providing another level of flexibility. As shown in user interface screenshots 510-540 of FIG. 5, maintenance of the SeqStudio Genetic Analyzer used in some embodiments of the present invention is intended to be simple and straightforward for the user, and instrument calibrations used in the genetic analyzer instrument used in exemplary embodiments of the invention described herein may be handled automatically.

As shown in FIG. 6, another level of convenience may be added by integrating wireless connectivity into the exemplary genetic analyzer instrument shown in FIG. 3. FIG. 6 shows how the SeqStudio genetic analyzer instrument may be accessible to the user in several different ways: via the onboard interface, a remote computer, or a mobile device app. In some embodiments of the invention, assays or experimental runs can be set up using either the onboard computer or by using PlateManager, the stand-alone software that operates within Thermo Fisher Connect or on a separate computer, as shown in 610. By using web browser-based software, access to run setup, plate maps, run conditions, and analysis settings My be immediately available via internet connection. Injection conditions, reinjections, and reordering of injections may be monitored and modified during the run as shown in 620, maximizing the ability to collect quality data from each plate. After data collection, the web browser-based suite of applications, including applications to measure microsatellite instability, allows accessible analysis in some embodiments of the present invention. Determination of DNA sequence variants, alignments, and fragment analysis are all available immediately upon completion of a run, in analysis step 630. Finally, the cloud connectivity 650 enables collaborators in different locations to monitor, access, share data information, and rapidly analyze the same data sets anytime post-run in sharing and collaboration step 640.

The SeqStudio Genetic Analyzer provides touchscreen usability via the instrument itself or via smartphone, tablet or other user device, allowing researches to collaborate and analyze data remotely as well as onsite. The exemplary genetic analyzer system discussed herein as used in some embodiments of the present invention is designed for both new and experienced users who need simple and affordable Sanger sequencing and fragment analysis, without compromising performance or quality.

FIG. 7 shows a flow diagram of a method 700 used in some embodiments of the present invention to determine the MSI status of a biological sample. In step 710, one or more DNA loci of the biological sample are selected to investigate for tumors which exhibit a high mutation rate and hence a high microsatellite instability.

Microsatellite instability (MSI) is a form of genomic instability due to reduced fidelity during the replication of DNA; this is thought to be caused by defects in DNA repair mechanisms. Defects in this biomolecular machinery is most easily observed by examining places in the DNA where there is a single nucleotide (one of the four possible nucleotides) repeated many times (a homopolymer); e.g., GGGGGGGGGGG is a 11-base repeat of Guanine. Extending this example, with damaged DNA repair mechanisms that often manifest in tumor cells, the section of DNA with the 11-base repeat of Guanine may be replicated as, for example, 10 bases or 5 bases or 13 bases instead of the normal 11 bases. Microsatellite instability analysis involves chemistries designed to examine several different regions in DNA at which there are homopolymers. These chemistries select out and amplify sections of DNA (an amplified fragment of DNA at specific DNA loci) that include each of the homopolymers of interest. Hence, again building on our 11-base Guanine example, normally, the amplified DNA at this locus would have a fragment size of, say, 20 bases (some number larger than 11 selected out by the chemistry). However, if DNA replication repair mechanisms are damaged, the replicated DNA may only have 10, for example, instead of the usual 11 Guanines so the amplified fragments will be of size 19 instead of 20. There are two ways to detect this situation using the technologies that are the subject of this disclosure: 1) analyze DNA from tumor tissue and normal tissue from the same person and compare the two or 2) analyze DNA from tumor tissue and compare to what is typically expected at each DNA locus of interest in the case where there is no damage to DNA repair mechanisms. Note that these concepts as well as the invention described in this disclosure also apply to non-homopolymer sections of DNA that consist of simple repeated sequences of nucleotides, e.g., ACACACAC or TATGTATGTATGTAGT, etc.

Thus, in step 710 particular DNA loci may be selected for the sensitivity of the loci to the cancer type under investigation as compared to other cancer types. A particular DNA locus (also referred to as a marker) may also be selected for the reliability of DNA amplification at that particular locus.

In step 720, each DNA locus is examined and one or more algorithms may be selected for each locus to determine whether that given locus is microsatellite unstable, MSU, or microsatellite stable, MSS. Embodiments of the present invention utilize a number of algorithms for determining whether or not a given DNA locus is MSU or MSS, including algorithms 1 through 11 below. In step 730, the selected algorithm(s) is executed for each selected DNA locus. In step 740, the overall MSI status is determined for the biological sample by combining the MSI results for each selected DNA locus.

FIG. 8 shows another embodiment of a method for assessing the microsatellite instability of a biological sample, which is first described above and in FIG. 7. The embodiment shown in FIG. 8 provides additional detail and alternate data analysis pathways elaborating on the embodiment of the microsatellite instability assessment method shown in FIG. 7. Starting at step 810 of FIG. 8, the CE fragment analysis data across all DNA loci for the biological sample is obtained. Each DNA locus, or marker, may be analyzed separately, as shown in step 805. Alternatively, the markers may be analyzed together as shown in step 815 of FIG. 8. If the markers are to be analyzed separately as in step 805, one or more signal features can be extracted for each marker in step 820, and one or more classification functions such as the exemplary classification functions described below can be applied to the extracted signal features to determine the MSI status of each marker in step 830.

1. A simple size threshold: Any fluorescence peaks appearing below the fragment size threshold (or above depending on the location for an MSS situation) is considered MSU. This is appropriate if there is only one DNA locus covered by a given dye and the number of nucleotides differs significantly between MSU and MSS DNA molecule situations.

2. Fragment size interval: If there are any fluorescence peaks appearing within a given fragment size interval (an interval on the DNA fragment size axis of the data), the DNA locus is considered MSU. This presumes that there is no overlap between the size intervals covering all the DNA loci using the same dye and the fluorescent peaks associated with MSU and MSS situations are also well separated.

3. Peak count within a given size interval: If the number of significant fluorescent peaks (significance determined by peak size) is above a threshold, the locus is considered MSU. This presumes that there is no overlap between the size intervals covering all the DNA loci using the same dye.

4. Relative peak count within a given size interval: If the number of significant fluorescent peaks (significance determined by peak size) deviates significantly from that expected for MSS, the locus is considered MSU. This presumes that there is no overlap between the size intervals covering all the DNA loci using the same dye.

5. Peak envelope peaks within a given size interval: If the number of envelope peaks is two or more, the locus is considered MSI-high. This presumes that there is no overlap between the size intervals covering all the DNA loci using the same dye.

6. Peak envelope separation within a given size interval: If the separation between the two largest envelope peaks deviates significantly from that of MSI-stable samples, the locus is considered MSI-high. This presumes that there is no overlap between the size intervals covering all the DNA loci using the same dye.

7. Peak pattern: Peak patterns can consist of two or more values among the following: peak amplitudes and/or locations along the fragment size axis; peak amplitudes and/or locations relative to the largest peak; peak envelope peak amplitudes, locations, and/or widths; peak metrics relative to peak envelope metrics;

8. Peak pattern deviations from normal: Peak patterns of (7) above relative to these patterns from normal tissue samples;

9. Peak pattern deviations from normal (non-cancer) population peak patterns: Peak patterns of (7) above relative to these patterns from nominal values for these patterns, such as the mean, median, z-score, etc., across a population of people without cancer.

10. Peak pattern deviations from normal relative to population deviations: A combination of (8) and (9) above where the metrics of (8) are compared to nominal values of these patterns across a population of people without cancer.

11. Difference signal patterns: In the case that data from a given person is available from both normal and tumor tissue, signal patterns described above can be computed on the difference between normalized data from tumor and normal tissue. In addition, other metrics derived from the difference signal can be used to characterize the difference signal at each locus. For example, asymmetry of the difference signal can be characterized by the difference between the center of mass of the positive peaks of the difference signal compared to the negative peaks. Other examples include the relative position of the difference signal maximum and minimum, the root-mean-square (RMS) values of positive compared to negative peaks, overall RMS value for the difference signal, etc.

For items (7) through (11), the algorithm for determining whether a DNA locus is MSS or MSU would consist of a suitable classification function that can process multi-dimensional vectors. For example, discriminant functions, multi-layer artificial neural networks, vector machines, etc. are examples of typical machine learning methods that can be applied. Alternatively, instead of pre-specifying signal features as outlined in items (7) to (10), deep learning methods can be applied to automatically learn the best signal features to distinguish MSU from MSS by using a large number of samples of CE fragment analysis data localized to the fragment size intervals of interest.

To make the overall assessment of MSI status in step 740 of FIG. 7, or starting at step 810 of FIG. 8, the MSI results for each DNA locus can be combined across all of the markers in the nucleic acid sample under investigation to assign an overall MSI status call in step 840 and 880. The following are exemplary methods for doing this when two or more DNA loci are used:

12. Fixed percentage level: If the percentage of DNA loci that are MSU is above a chosen threshold, the overall assignment is MSI-high (or MSI-low if the percentage of DNA loci that are MSU are below the first chosen threshold but above a second predetermined threshold) and MSS if the percentage of DNA loci that are MSU are below both of these thresholds.

13. Weighted sum: In one embodiment of the invention, a weighted sum across DNA loci can be calculated after assigning MSU loci an exemplary value of 1 and MSS loci an exemplary value of 0; the overall assessment can be assigned MSI-high if the weighted sum across loci exceeds a threshold. Linear discriminant functions are an exemplary way to determine the weightings.

14. Non-linear classification: As shown in FIG. 8 at steps 820 and 830, the MSI results across DNA loci can alternatively be combined in a non-linear fashion to determine an overall assessment of MSI status in steps 840 and 880. An exemplary way to determine the non-linear classification function is to train a multi-layer artificial neural network to make the assignment.

Standard 3-layer artificial neural networks, trained with customary backpropagation techniques known in the art to minimize cross entropy, have been found to provide adequate accuracy in distinguishing MSU from MSS cases.

15. Direct to overall assessment: Instead of pre-assessing each DNA locus for MSI status as in step 805, the markers may be analyzed together in step 815 and the signal features expressed in items (7) to (11) can be combined across DNA loci as shown in step 860 and used to generate one or more classification functions that directly assigns the overall MSI status as shown in step 870 of FIG. 8. For example, suppose marker A used 3 signal features to determine whether it is MSU or MSS and marker B used 4 features for this. In one embodiment of the present invention, the features from both markers can be combined into a 7-dimensional feature vector to feed into an artificial intelligence generated classifier, such as an artificial neural network trained to map this vector to an overall MSI status call. Instead of an explicit algorithm to combine information across markers the combining of this information becomes inherent in the training of this artificial neural network classifier. Alternatively, artificial intelligence generated classifiers may be implemented via deep learning methods, which can be used as described above except that data across DNA loci are combined in the analysis either after the markers are analyzed separately in step 805 or together, as shown in step 815. The signal features are fed directly into a deep learning neural network for mapping directly into an MSI status call, as shown in step 850 of FIG. 8. For example, signals from each locus can be concatenated into one large signal vector and fed into a deep learning network to map them into an overall MSI status call at step 880.

Some embodiments of the present invention comprise methods for using one or more anti-tumor drugs to treat tumor patients. In particular embodiments, one or more of the methods, computer program products, systems, or kits disclosed herein are used to determine microsatellite instability of tumor cells in a biological sample obtained from a patient. Then, if microsatellite instability is determined to be high, the one or more anti-tumor drugs are administered to the patient to treat the tumor.

Systems, apparatus, and methods described herein may be implemented using a computer program product tangibly embodied in an information carrier, e.g., in a non-transitory machine-readable storage device, for execution by a programmable processor; and the method steps described herein, including one or more of the steps of the methods in FIG. 6, FIG. 7 and FIG. 8 and alternative embodiments may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

FIG. 9 illustrates components of one embodiment of an environment 900 in which the invention may be practiced. Not all the components may be required to practice the invention, and variations in the arrangement and type of the components may be made without departing from the spirit or scope of the invention. As shown, the system 900 includes one or more Local Area Networks (“LANs”)/Wide Area Networks (“WANs”) 912, one or more wireless networks 910, one or more wired or wireless client devices 906, mobile or other wireless client devices 902-906, servers 907-909, and may include or communicate with one or more data stores or databases. Various of the client devices 902-906 may include, for example, desktop computers, laptop computers, set top boxes, tablets, monitors, cell phones, smart phones, devices for interfacing with, or viewing dashboards or analytics relating to, genetic analysis related systems or entities, etc. The servers 907-909 can include, for example, one or more application servers, content servers, search servers, database servers, database management or SQL servers, other servers relating to genetic analysis related systems, etc.

FIG. 10 illustrates a block diagram of an electronic device 1100 that can implement one or more aspects of genetic analysis related systems and methods according to embodiments of the invention. Instances of the electronic device 1100 may include servers, e.g., servers 907-909, and client devices, e.g., client devices 902-906.

FIG. 11 shows an example of a computer system 1100, one or more of which may provide one or more of the components of, or alternatives to computer 103 of FIG. 1. Computer system 1100 executes instruction code contained in a computer program product 1122 comprising genetic analyzer program 1123 (which may, for example, comprise CE data analyzer program 104 of the computer program product 102 of the embodiment of FIG. 1.) Computer program product 1122 comprises executable code in an electronically readable medium that may instruct one or more computers such as computer system 1100 to perform processing that accomplishes the exemplary method steps performed by the embodiments referenced herein. The electronically readable medium may be any non-transitory medium that stores information electronically and may be accessed locally or remotely, for example via a network connection. In alternative embodiments, the medium may be transitory. The medium may include a plurality of geographically dispersed media each configured to store different parts of the executable code at different locations and/or at different times. The executable instruction code in an electronically readable medium directs the illustrated computer system 1100 to carry out various exemplary tasks described herein. The executable code for directing the carrying out of tasks described herein would be typically realized in software. However, it will be appreciated by those skilled in the art, that computers or other electronic devices might utilize code realized in hardware to perform many or all the identified tasks without departing from the present invention. Those skilled in the art will understand that many variations on executable code may be found that implement exemplary methods within the spirit and the scope of the present invention.

The code or a copy of the code contained in computer program product 1100 may reside in one or more storage persistent media (not separately shown) communicatively coupled to system 1100 for loading and storage in persistent storage device. In general, the electronic device 1100 can include a processor/CPU 1102, memory 1130, a power supply 1106, and input/output (I/O) components/devices 1140, e.g., microphones, speakers, displays, touchscreens, keyboards, mice, keypads, microscopes, GPS components, etc., which may be operable, for example, to provide graphical user interfaces, dashboards, etc.

A user may provide input via a touchscreen of an electronic device 1100. A touchscreen may determine whether a user is providing input by, for example, determining whether the user is touching the touchscreen with a part of the user's body such as his or her fingers. The electronic device 1100 can also include a communications bus 1104 that connects the aforementioned elements of the electronic device 1100. Network interfaces 1114 can include a receiver and a transmitter (or transceiver), and one or more antennas for wireless communications.

The processor 1102 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 1130, which can include Random Access Memory (RAM) 1112 and Read Only Memory (ROM) 1132, can be enabled by one or more of any type of memory device, e.g., a primary (directly accessible by the CPU) or secondary (indirectly accessible by the CPU) storage device (e.g., flash memory, magnetic disk, optical disk, and the like). The ROM 1132 can also include Basic Input/Output System (BIOS) 1120 of the electronic device.

The RAM can include an operating system 1121, data storage 1124, which may include one or more databases, and programs and/or applications 1122 and a genetic analyzer program 1123. The genetic analyzer program 1123 is intended to broadly include all programming, applications, algorithms, software and other and tools necessary to implement or facilitate methods and systems according to embodiments of the invention. Elements of the genetic analyzer program 1123 program may exist on a single server computer or be distributed among multiple computers, servers, devices or entities, or sites. Moreover, those skilled in the art will appreciate that in addition to storing computer program product 1122 for carrying out processing described herein, memory 1130 may be configured to store the various data elements referenced and illustrated herein.

The power supply 1106 contains one or more power components and facilitates supply and management of power to the electronic device 1100.

The input/output components, including Input/Output (I/O) interfaces 1140, can include, for example, any interfaces for facilitating communication between any components of the electronic device 1100, components of external devices (e.g., components of other devices of the network or system 1100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 1140 and the bus 1104 can facilitate communication between components of the electronic device 1100, and in an example can ease processing performed by the processor 1102.

Where the electronic device 1100 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications.

Any computing device capable of sending, receiving, and processing data over a wired and/or a wireless network may act as a server, such as in facilitating aspects of implementations of genetic analyzer related systems and methods according to embodiments of the invention. Devices acting as a server may include devices such as dedicated rack-mounted servers, desktop computers, laptop computers, set top boxes, integrated devices combining one or more of the preceding devices, etc.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of systems and methods according to embodiments of the invention. One or more servers may, for example, be used in hosting a Web site utilized in embodiments of the present invention. One or more servers may host a variety of sites, such as, for example, business sites, informational sites, social networking sites, educational sites, wilds, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of systems and methods according to embodiments of the invention. Content may include, for example, text, images, audio, video, and the like.

In example aspects of genetic analyzer systems and methods according to embodiments of the invention, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, monitors, sensor-equipped devices, laptop computers, set top boxes, wearable computers, integrated devices combining one or more of the preceding devices, and the like.

Client devices may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, GPS or other location-aware capability, and a 2D or 3D touch-sensitive color screen on which both text and graphics may be displayed.

Client devices, such as client devices 1002-1006, for example, as may be used in example systems and methods according to embodiments of the invention, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as viewing or interacting with analytics or dashboards, interacting with genetic analyzer instruments, methods or systems used in embodiments of the present invention, browsing webpages, using a web search engine, interacting with various apps stored on a smart phone, sending and receiving messages via email, SMS, or MMS, playing games, receiving advertising, watching locally stored or streamed video, or participating in social networks. In example aspects of genetic analyzer systems and methods according to embodiments of the invention, one or more networks, such as networks 1010 or 1012, for example, may couple servers and client devices with other computing devices, including through wireless network to client devices. A network may be enabled to employ any form of computer readable media for communicating information from one electronic device to another. A network may include the Internet in addition to Local Area Networks (LANs), Wide Area Networks (WANs), direct connections, such as through a Universal Serial Bus (USB) port, other forms of computer-readable media, or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 1010, as in example genetic analysis related systems and methods according to embodiments of the invention, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, 5G and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, AppleTalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in size), padding (may be composed of all zeros and includes a number of bits such that the header ends on a 32 bit boundary). The number of bits for each of the above may also be higher or lower.

A “content delivery network” or “content distribution network” (CDN), as may be used in example systems and methods according to embodiments of the invention, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's Web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

One embodiment of the present invention includes systems, methods, and a non-transitory computer readable storage medium or media tangibly storing computer program logic capable of being executed by a computer processor.

Those skilled in the art will appreciate computer system 1100 illustrates just one example of a system in which a computer program product in accordance with an embodiment of the present invention may be implemented. To cite but one example of an alternative embodiment, execution of instructions contained in a computer program product in accordance with an embodiment of the present invention may be distributed over multiple computers, such as, for example, over the computers of a distributed computing network.

Embodiments of the present invention include the following:

### Embodiment 1

A method of identifying microsatellite instability in a biological sample comprising:

obtaining a plurality of signals by detecting fluorescence of fragments comprising nucleic acid sequences obtained using the biological sample wherein each signal corresponds to one of a plurality of different microsatellite loci;

determining one or more signal features for each of the plurality of signals; and

applying one or more classifiers to one or more of the signal features of the plurality of microsatellite loci to identify whether the biological sample is microsatellite instability high, microsatellite instability low, or microsatellite stable.

### Embodiment 2

The method of embodiment 1, further comprising applying one or more classifiers to one or more signal features corresponding to the signal for each individual microsatellite locus in the plurality of different microsatellite loci to identify whether each individual microsatellite locus is microsatellite unstable or microsatellite stable and combining these determinations across loci to determine a microsatellite status of the biological sample.

### Embodiment 3

The method of embodiment 1 or embodiment 2, wherein the applying one or more classifiers comprise comparing a signal feature derived from the biological sample and a signal feature derived from one or more samples of non-cancerous tissue.

### Embodiment 4

The method of embodiment 1 or embodiment 2, wherein at least one classifier comprises a fragment size threshold.

### Embodiment 5

The method of embodiment 1 or embodiment 2, wherein at least one classifier comprises a fragment size interval.

### Embodiment 6

The method of embodiment 1 or embodiment 2, wherein applying the one or more classifiers comprises evaluating a peak count within a specified size interval.

### Embodiment 7

The method of embodiment 1 or embodiment 2, wherein applying the one or more classifiers comprises a evaluating a relative peak count between tumor and normal tissues within a specified size interval.

### Embodiment 8

The method of embodiment 1 or embodiment 2, wherein applying the one or more classifiers comprises evaluating a peak envelope count within a specified size interval.

### Embodiment 9

The method of embodiment 1 or embodiment 2, wherein applying the one or more classifiers comprises evaluating a peak envelope separation within a specified size interval.

### Embodiment 10

The method of embodiment 1 or embodiment 2, wherein applying the one or more classifiers comprises evaluating a peak envelope separation within a specified size interval.

### Embodiment 11

The method of embodiment 1 or embodiment 2, wherein applying the one or more classifiers comprises evaluating a shift in one or more peak locations within a specified size interval.

### Embodiment 12

The method of embodiment 1 or embodiment 2, wherein applying the one or more classifiers comprises analyzing a peak pattern input.

### Embodiment 13

The method of embodiment 12, wherein the peak pattern input comprises two or more values of: peak amplitudes along the fragment size axis, peak locations along the fragment size axis; peak amplitudes relative to a largest peak, peak locations relative to a largest peak, peak envelope peak amplitudes, peak envelope peak locations, peak envelope peak widths, or peak metrics relative to peak envelope metrics.

### Embodiment 14

The method of embodiment 2, further comprising:

assigning the biological sample a high microsatellite instability status when a percentage of the microsatellite loci is determined to be microsatellite unstable is above a first predetermined threshold, a low microsatellite instability status if the percentage of microsatellite loci is determined to be microsatellite unstable is above a second predetermined threshold but below the first determined threshold, or a microsatellite stable status if the percentage of microsatellite loci determined to be microsatellite unstable is below the second predetermined threshold.

### Embodiment 15

The method of embodiment 2, further comprising:

analyzing the signal features to assign either a stable value or an unstable value to each of the microsatellite loci;

calculating a weighted sum across the assigned stable and unstable values of the microsatellite loci; and

assigning the biological sample a high microsatellite instability status if the weighted sum across the microsatellite loci exceeds a first predetermined threshold, assigning the biological sample a low microsatellite instability status if the weighted sum across the microsatellite loci exceeds a second predetermined threshold but not the first predetermined threshold, or a microsatellite stable status if the weighted sum across the microsatellite loci is less than the second predetermined threshold.

### Embodiment 16

The method of embodiment 15, wherein the weighted sum is calculated using one or more classification functions which map the plurality of signal features to three distinct output values.

### Embodiment 17

A method for identifying microsatellite instability in a biological sample, comprising:

obtaining a plurality of signals by detecting fluorescence of fragments comprising nucleic acid sequences amplified from the biological sample, the nucleic acid sequences corresponding to a plurality of different microsatellite loci wherein each signal corresponds to one of a plurality of different microsatellite loci; and

analyzing the plurality of signals using one or more classifiers to identify whether the biological sample has high microsatellite instability, low microsatellite instability, or is microsatellite stable.

### Embodiment 18

The method of embodiment 17, wherein the classifier comprises a non-linear classification function.

### Embodiment 19

The method of embodiment 18, wherein the non-linear classification function comprises a multi-layer artificial neural network.

### Embodiment 20

The method of embodiment 17, wherein the classifier comprises a deep learning neural network.

### Embodiment 21

A computer program product comprising:

executable code stored in a non-transitory computer readable medium executable on one or more computer processors to identify microsatellite instability in a biological sample, the executable code comprising one or more computer readable instructions for:
obtaining a plurality of signals from a capillary electrophoresis genetic analysis instrument, wherein the signals are detected from fluorescence of fragments comprising nucleic acid sequences amplified from the biological sample via polymerase chain reaction, the nucleic acid sequences corresponding to a plurality of different microsatellite loci wherein each signal corresponds to one of a plurality of different microsatellite loci; and
analyzing each of the plurality of signals using one or more classifiers to identify whether the biological sample has a microsatellite instability high, microsatellite instability low, or microsatellite stable status.

### Embodiment 22

The computer program product of embodiment 21, wherein at least one classifier comprises an artificial intelligence generated classifier.

### Embodiment 23

A system for identifying microsatellite instability in a biological sample using a capillary electrophoresis genetic analysis instrument, comprising:

one or more computer processors connected to a non-transitory computer readable medium storing one or more computer readable instructions that, when executed by the one or more computer processors:
a. obtain a plurality of signals from a capillary electrophoresis genetic analysis instrument, wherein the signals are detected from fluorescence of fragments comprising nucleic acid sequences amplified from the biological sample via polymerase chain reaction, the nucleic acid sequences corresponding to a plurality of different microsatellite loci wherein each signal corresponds to one of a plurality of different microsatellite loci; and
b. analyze each of the plurality of signals using one or more classifiers to identify whether the biological sample has a microsatellite instability high, microsatellite instability low, or microsatellite stable status;

a memory connected to at least one of the one or more processors for storing one or more of the signal features; and

a user device display connected to the memory and configured to display one or more of the signal features.

### Embodiment 24

The system of embodiment 23, wherein at least one classifier comprises an artificial intelligence generated classifier.

### Embodiment 25

A kit for identifying microsatellite instability in a biological sample, the kit comprising:

a plurality of polymerase chain reaction (PCR) primers configured to flank a plurality of microsatellite loci of a biological sample such that, when the PCR primers and the biological sample are combined and subjected to an amplification process, fluorescently labeled DNA fragments are generated comprising the plurality of microsatellite loci, wherein at least some of the plurality of microsatellite loci are different from others of the plurality of microsatellite loci; and

a computer program product embedded in a non-transitory computer readable medium comprising executable instruction code that, when executed by one or more processors causes the one or more processors to perform processing comprising:
using fluorescent data obtained from the plurality of fluorescently labelled microsatellite loci to classify microsatellite instability of the biological sample.

### Embodiment 26

The kit of embodiment 25, wherein execution of the executable instruction code causes the one or more processors to perform processing comprising:

applying one or more locus-specific algorithms to the fluorescent data to generate locus-specific results classifying microsatellite instability of corresponding specific loci of the plurality of microsatellite loci; and

using the locus-specific results to classify the microsatellite instability of the biological sample.

### Embodiment 27

A method of treating a patient having a tumor with an anti-tumor drug, the anti-tumor drug being more likely to be effective when cells of the tumor exhibit high microsatellite instability, the method comprising:

obtaining a biological sample from the patient, the biological sample comprising cells of the tumor;

determining whether the cells of the tumor exhibit high microsatellite instability using the method of any one of embodiments 1-20; and if the microsatellite instability of the cells of the tumor is determined to be high, administering the anti-tumor drug to the patient to treat the tumor.

### Embodiment 28

A method of treating a patient having a tumor with an anti-tumor drug, the anti-tumor drug being more likely to be effective when cells of the tumor exhibit high microsatellite instability, the method comprising:

obtaining a biological sample from the patient, the biological sample comprising cells of the tumor;

determining whether the cells of the tumor exhibit high microsatellite instability using the computer program product of any one of embodiments 21-22; and

if the microsatellite instability of the cells of the tumor is determined to be high, administering the anti-tumor drug to the patient to treat the tumor.

### Embodiment 29

A method of treating a patient having a tumor with an anti-tumor drug, the anti-tumor drug being more likely to be effective when cells of the tumor exhibit high microsatellite instability, the method comprising:

obtaining a biological sample from the patient, the biological sample comprising cells of the tumor;

determining whether the cells of the tumor exhibit high microsatellite instability using the system of any one of embodiments 23-24; and

if the microsatellite instability of the cells of the tumor is determined to be high, administering the anti-tumor drug to the patient to treat the tumor.

### Embodiment 30

A method of treating a patient having a tumor with an anti-tumor drug, the anti-tumor drug being more likely to be effective when cells of the tumor exhibit high microsatellite instability, the method comprising:

obtaining a biological sample from the patient, the biological sample comprising cells of the tumor;

determining whether the cells of the tumor exhibit high microsatellite instability using the kit of any one of embodiments 25-26; and

if the microsatellite instability of the cells of the tumor is determined to be high, administering the anti-tumor drug to the patient to treat the tumor.

### Embodiment 31

A method of treating a patient having a tumor with an anti-tumor drug, the anti-tumor drug being more likely to be effective when cells of the tumor exhibit high microsatellite instability, the method comprising:

obtaining a plurality of biological samples from the patient, at least one biological sample comprising cells of the tumor and at least one biological sample comprising normal cells;

determining whether the cells of the tumor, relative to the normal cells, exhibit high microsatellite instability using the method of any one of embodiments 1-20; and if the microsatellite instability of the cells of the tumor is determined to be high, administering the anti-tumor drug to the patient to treat the tumor.

While the present invention has been particularly described with respect to the illustrated embodiments, it will be appreciated that various alterations, modifications and adaptations may be made based on the present disclosure and are intended to be within the scope of the present invention. While the invention has been described in connection with what are presently considered to be the most practical and preferred embodiments, it is to be understood that the present invention is not limited to the disclosed embodiments but, on the contrary, is intended to cover various modifications and equivalent arrangements included within the scope of the underlying principles of the invention as described by the various embodiments referenced above and below.

What is claimed is:

1. A method of identifying microsatellite instability in a biological sample comprising:
obtaining a plurality of signals by detecting fluorescence of fragments comprising nucleic acid sequences obtained using the biological sample wherein each signal corresponds to one of a plurality of different microsatellite loci;
determining one or more signal features for each of the plurality of signals; and
applying one or more classifiers to one or more of the signal features of the plurality of microsatellite loci to identify whether the biological sample is microsatellite instability high, microsatellite instability low, or microsatellite stable.

2. The method of claim 1, further comprising applying one or more classifiers to one or more signal features corresponding to the signal for each individual microsatellite locus in the plurality of different microsatellite loci to identify whether each individual microsatellite locus is microsatellite unstable or microsatellite stable and combining these determinations across loci to determine a microsatellite status of the biological sample.

3. The method of claim 1, wherein the applying one or more classifiers comprise comparing a signal feature derived from the biological sample and a signal feature derived from one or more samples of non-cancerous tissue.

4. The method of claim 1, wherein at least one classifier comprises a fragment size threshold or a fragment size interval.

5. The method of claim 1, wherein applying the one or more classifiers comprises evaluating a peak count, or a relative peak count between tumor and normal tissues within a specified size interval.

6. The method of claim 1, wherein applying the one or more classifiers comprises evaluating a peak envelope count or a peak envelope separation within a specified size interval.

7. The method of claim 1, wherein applying the one or more classifiers comprises evaluating a shift in one or more peak locations within a specified size interval.

8. The method of claim 1, wherein applying the one or more classifiers comprises analyzing peak pattern input comprising two or more values of: peak amplitudes along the fragment size axis, peak locations along the fragment size axis; peak amplitudes relative to a largest peak, peak locations relative to a largest peak, peak envelope peak amplitudes, peak envelope peak locations, peak envelope peak widths, or peak metrics relative to peak envelope metrics.

9. The method of claim 8, further comprising measuring the deviations of peak pattern input for the biological sample relative to peak pattern input from normal tissue samples.

10. The method of claim 8, further comprising measuring the deviations of peak pattern input for the biological sample relative to nominal peak pattern values calculated across a population of individuals without cancer.

11. The method of claim 8, further comprising measuring the deviations of peak pattern input for the biological sample relative to peak pattern input from normal tissue samples, as compared to nominal peak pattern values calculated across a population of individuals without cancer.

12. The method of claim 8, further comprising computing a difference signal between normalized data from tumor cells of the biological sample and normalized data from normal non-tumor cells of the biological sample and deriving peak patterns based on these difference signals.

13. The method of claim 2, further comprising:
assigning the biological sample a high microsatellite instability status when a percentage of the microsatellite loci is determined to be microsatellite unstable is above a first predetermined threshold, a low microsatellite instability status if the percentage of microsatellite loci is determined to be microsatellite unstable is above a second predetermined threshold but below the first determined threshold, or a microsatellite stable status if the percentage of microsatellite loci determined to be microsatellite unstable is below the second predetermined threshold.

14. The method of claim 2, further comprising:
analyzing the signal features to assign either a stable value or an unstable value to each of the microsatellite loci;
calculating a weighted sum across the assigned stable and unstable values of the microsatellite loci; and
assigning the biological sample a high microsatellite instability status if the weighted sum across the microsatellite loci exceeds a first predetermined threshold, assigning the biological sample a low microsatellite instability status if the weighted sum across the microsatellite loci exceeds a second predetermined threshold but not the first predetermined threshold, or a microsatellite stable status if the weighted sum across the microsatellite loci is less than the second predetermined threshold.

15. The method of claim 14, wherein the weighted sum is calculated using one or more classification functions which map the plurality of signal features to three distinct output values.

16. A method for identifying microsatellite instability in a biological sample, comprising:
obtaining a plurality of signals by detecting fluorescence of fragments comprising nucleic acid sequences amplified from the biological sample, the nucleic acid sequences corresponding to a plurality of different microsatellite loci wherein each signal corresponds to one of a plurality of different microsatellite loci; and
analyzing the plurality of signals using one or more classifiers to identify whether the biological sample has high microsatellite instability, low microsatellite instability, or is microsatellite stable.

17. The method of claim 16, wherein the classifier comprises a non-linear classification function.

18. The method of claim 17, wherein the non-linear classification function comprises a multi-layer artificial neural network.

19. The method of claim 16, wherein the classifier comprises a deep learning neural network.

20. A computer program product comprising executable code stored in a non-transitory computer readable medium executable on one or more computer processors to identify microsatellite instability in a biological sample, the executable code comprising one or more computer readable instructions for:
obtaining a plurality of signals from a capillary electrophoresis genetic analysis instrument, wherein the signals are detected from fluorescence of fragments comprising nucleic acid sequences amplified from the biological sample via polymerase chain reaction, the nucleic acid sequences corresponding to a plurality of different microsatellite loci wherein each signal corresponds to one of a plurality of different microsatellite loci; and
analyzing each of the plurality of signals using one or more classifiers to identify whether the biological sample has a microsatellite instability high, microsatellite instability low, or microsatellite stable status.

21. The computer program product of claim 20, wherein at least one classifier comprises an artificial intelligence generated classifier.

22. A system for identifying microsatellite instability in a biological sample using a capillary electrophoresis genetic analysis instrument, comprising:
one or more computer processors connected to a non-transitory computer readable medium storing one or more computer readable instructions that, when executed by the one or more computer processors:
obtain a plurality of signals from the capillary electrophoresis genetic analysis instrument, wherein the signals are detected from fluorescence of fragments comprising nucleic acid sequences amplified from the biological sample via polymerase chain reaction, the nucleic acid sequences corresponding to a plurality of different microsatellite loci wherein each signal corresponds to one of a plurality of different microsatellite loci; and
analyze each of the plurality of signals using one or more classifiers to identify whether the biological sample has a microsatellite instability high, microsatellite instability low, or microsatellite stable status;
a memory connected to at least one of the one or more processors for storing one or more of the signal features; and
a user device display connected to the memory and configured to display one or more of the signal features.

23. A kit for identifying microsatellite instability in a biological sample, the kit comprising:
a plurality of polymerase chain reaction (PCR) primers configured to flank a plurality of microsatellite loci of a biological sample such that, when the PCR primers and the biological sample are combined and subjected to an amplification process, fluorescently labeled DNA fragments are generated comprising the plurality of microsatellite loci, wherein at least some of the plurality of microsatellite loci are different from others of the plurality of microsatellite loci; and
a computer program product embedded in a non-transitory computer readable medium comprising executable instruction code that, when executed by one or more processors causes the one or more processors to perform processing comprising:
using fluorescent data obtained from the plurality of fluorescently labelled microsatellite loci to classify microsatellite instability of the biological sample.

24. The kit of claim 23, wherein execution of the executable instruction code causes the one or more processors to perform processing comprising:
applying one or more locus-specific algorithms to the fluorescent data to generate locus-specific results classifying microsatellite instability of corresponding specific loci of the plurality of microsatellite loci; and
using the locus-specific results to classify the microsatellite instability of the biological sample.
